(19) United States
(12) Patent Application Publication
(10) Pub. No.: US 2023/0078844 A1
(43) Pub. Date: Mar. 16, 2023

(54) Illumination System with Etendue-Squeezing Module and Method Thereof

(71) Applicant: Apollo Medical Optics, Ltd., Taipei (TW)

(72) Inventors: Tuan-Shu HO, Taipei (TW); Ming-Rung TSAI, Taipei (TW); Chih-Wei LU, Taipei (TW)

(21) Appl. No.: 17/796,275

(22) PCT Filed: Feb. 1, 2021
(86) PCT No.: PCT/US21/16107

§ 371 Date: Jul. 29, 2022

Related U.S. Application Data
(60) Provisional application No. 62/968,170, filed on Jan. 31, 2020.

(51) Int. Cl. A61B 5/00

(52) U.S. Cl. CPC A61B 5/0066; A61B 5/441; G01B 9/02015

(57) ABSTRACT

Provided herein are devices and systems comprising a light source which provides a beam to an optical module via a multimode fiber, an interference objective module outputs the beam processed by the optical module and collects interference signals from a sample; and a detector which detects the interference signals from the interference objective module wherein the optical module comprises an etendue squeezing component configured to slice the beams to at least two sub-beams and homogenize the sub-beams to an illumination field and match the shapes of the illumination field with the region of interest.

### BACKGROUND OF THE INVENTION

According to the statistic of World Health Organization, skin cancer has grown year-on-year in the past decade globally, closely related to lifestyle, aging society, and the destruction of the global ozone layer. Skin cancers are cancers that arise from the skin. They are due to the development of abnormal cells that have the ability to invade or spread to other parts of the body.

Cellular-resolution optical imaging techniques like reflectance confocal microscopy (RCM) are emerging to assist diagnosis of skin cancers and other skin diseases. However, RCM is usually designed with comparably low axial resolution to achieve useful penetration depth in turbid tissue. With broadband light sources and high-NA optics, optical coherence tomography (OCT) offers much better axial resolution comparing to RCM, and is thus an efficient tool to reveal the cross-sectional microstructure near the dermal-epidermal junction.

The advantage of such high-resolution OCT for the diagnosis of skin diseases is recently reported. With dynamic focusing between the objective lens and sample, good lateral resolution can be maintained through >300-μm depth in B-scan. However, due to multiple scattering in turbid tissue, single OCT images are often severely corrupted by coherent crosstalk, making small organelles like clusters of melanin difficult to be identified, especially by OCT with spatially-coherent sources.

### SUMMARY OF THE INVENTION

The present invention relates to an interference device/system employing an etendue squeezing module to improve the interference image quality. The present invention is also related to a method of detecting interference signals by applying etendue squeezing method to generate high quality cross-sectional (B-scan) images and en-face images (E-scan) of a sample.

The present invention provides an interference system comprising a light source which provides a beam to an optical module via a multimode fiber; an interference objective module outputs the beam processed by the optical module and collects interference signals from a sample; and a detector which detects the interference signals from the interference objective module wherein the optical module comprises an etendue squeezing component configured to slice the beams to at least two sub-beams and homogenize the sub-beams to an illumination field and match the shapes of the illumination field with the region of interest.

In another aspect provides a method of detecting interference signals comprising the steps providing a beam from a light source; reducing light divergence angle of the beam from the light source by a first lens group; slicing the beam to at least two sub-beams by an optical slicer; homogenizing the sub-beams and matching the shapes of the illumination field with the region of interest and projecting on a sample; and detecting interference signals from the sample.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 shows embodiment of the invention interference device/system.

FIG. 2 shows an embodiment of the invention interference device/system.

FIG. 3(a)˜(b) shows interference images of a human skin from the invention interference device.

FIG. 4 illustrates an embodiment of the invention device/system with a line of illumination filed.

FIG. 5 illustrates an embodiment of the invention device/system with an area of illumination filed.

FIG. 6(a)˜(d) shows interference images of a human skin from the invention interference device/system.

### DETAILED DESCRIPTION OF THE INVENTION

Biomedical imaging systems with spatial resolution around one micron can resolve cellular structure and provide important information for clinical diagnosis and treatment. Cross-sectional imaging (B-scan) is of special interest since it provides relative morphological information between cell layers. Optical imaging systems with high numerical aperture (NA) are possible to achieve in vivo cellular imaging.

Some optical imaging systems with a small-etendue light source of V number around 60 can achieve efficient en face imaging and B-scan imaging. However, the B-scan rate may be still slow since most of the light was loss in B-scan mode due to the finite etendue of the light source. Besides, some coherent artifacts are remained since the spatial coherence area is still large under the partially coherent line-field illumination scheme. In accordance with the practice of this invention there is provided a simple and efficient way to narrow the illumination linewidth (e.g., narrow down to around 5 μm), which is close to the typical thickness of a histological slice.

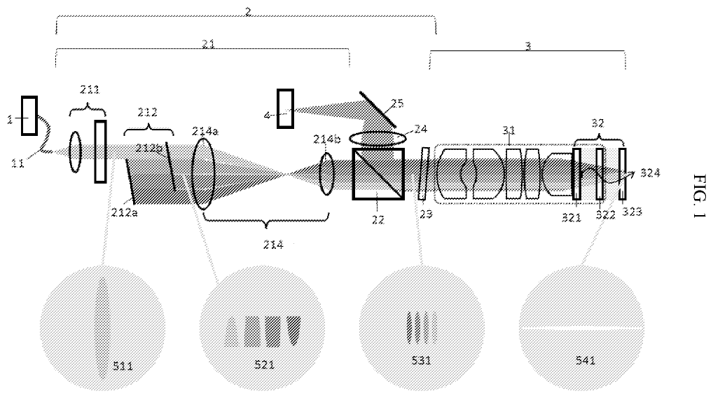

In order to improve the interference image quality and decrease artifacts on the interference images, the present invention provides an embodiment of an interference system illustrated in FIG. 1. In some embodiments provide an interference system/device comprising a light source 1 which provides a beam to an optical module 2 via a multimode fiber 11; an interference objective module 3 outputs the beam from the optical module 2 and collects interference signals during a measurement; and a detector 4 which detects the interference signals from the interference objective module 3 wherein the optical module 2 comprises an etendue squeezing component 21 configured to reduce light divergence angle of the beam from the light source 1 with a squeezing ratio of N, wherein N is at least 2. In certain embodiments, N is 2 to 16, 2 to 14, 2 to 12, 2 to 10, or 2 to 8, or other suitable ranges recognized by a person skilled in the art to improve the image quality of the interference images. In certain embodiments, N is 2 to 8. In some embodiments, the squeezing ratio (i.e., N) is defined as: N=NA_{fφf}/NAₒhᵢ. Thus, in order to efficiently couple the light from the multimode fiber 11 of numerical aperture NA_{f} and emitting diameter φ_{f} into an objective 31 with NA₀ and with an illumination linewidth of hᵢ, the required squeezing ratio can be estimated as N=NA_{fφf}/NAₒhᵢ. For example, in some embodiments, when NA=0.8 of the objective 31, a squeezing ratio N=4 is chosen (per the calculation above) to achieve illumination linewidth around 5 μm.

To achieve efficient B-scan with a multimode light source, extra optical loss due to the mismatch between the region of interest and illumination area must be considered. For example, for an optical coherence tomography (OCT) device with a two-dimensional detection (e.g., a 2-D camera), an efficient speckle suppression method is to compound close adjacent B-scans along the direction orthogonal to the imaging plane. These B-scans are synchronized acquired, demodulate and averaged to suppress the speckle noise, since the speckle patterns are not so correlated. To minimize the loss of spatial resolution (i.e. blurring) with acceptable speckle contrast, the virtual slice thickness usually chosen to be 3˜6 μm, which is close to typical histologic slice. For example, with light emitted from a multimode fiber with core size of 106 μm and NA=0.22, the minimal linewidth of illumination field (with little loss) is around 20˜40 μm even with a high NA objective lens. With the large difference between the linewidth of illumination field and target virtual slice thickness, many photons are wasted in B-scan mode, and B-scan rate is limited by photon noise. In some instances, the light emitted from a multimode fiber may be randomly-polarized, and 50% of photon may be loss as firstly passing through the polarizing beamsplitter, and the light is then linearly polarized.

In some embodiments provide an interference device (or a system comprising the device) comprising a light source which provides a beam to an optical module via a multimode fiber; an interference objective module outputs the beam processed by the optical module and collects interference signals from a sample; and a detector which detects the interference signals from the interference objective module wherein the optical module comprises an etendue squeezing component configured to slice the beams to at least two sub-beams and homogenize the sub-beams to an illumination field and match the shapes of the illumination field with a region of interest.

As shown in FIG. 1, the etendue squeezing component 21 comprises a first lens group 211 configured to reduce the light divergence angle of the beam from the light source 1 to provide an illumination spot 511. The first lens group 211, in some instances, comprises anamorphic optics; and the spot size along a first direction is larger comparing to a second direction perpendicular to the first direction. For example, the light emitted from multimode fiber 11 is firstly collimated by the first lens group 211, then directed to the squeezing optics. In some embodiments, the first lens group comprises a projection lenses, a collimator, an anamorphic collimator, a circular symmetric lens, or combinations thereof.

In some embodiments, the etendue squeezing component comprises a first lens group configured to reduce light divergence angle of the beam from the light source and project to an optical slicer wherein said optical slicer slices the processed beam to at least two sub-beams to be further processed by a second lens group before entering to said interference objective module via a polarization beam splitter. In some embodiments, the second lens group comprises a beam reducing optics. In some embodiments, the number of sub-beams is decided (determined) by a squeezing ratio of N. In certain embodiments, the squeezing ratio of N is 2 to 16, 2 to 14, 2 to 10, or 2 to 8.

The etendue squeezing component 21 further comprises an optical slicer 212 configured to slice the beam processed by the first lens group 211 into at least two sub-beams, in which the number of sub-beams depends on the squeezing ratio of N. In certain embodiments, squeezing ratio of N is 4. In some embodiments, the optical slicer 212 is selected from a group consisting of reflective mirrors, a prism, a wedge, and combinations thereof. A person skilled in the art would readily choose appropriate and suitable light slicer to achieve the same optical slicing function. In some embodiments, the optical slicer 212 comprises two parallel reflective mirrors 212a and 212b, each of them with one sharp edge. The beam enters the optical slicer 212 by the sharp edge of the first mirror 212a. After several reflections (e.g., at each two reflections), the beam laterally offset by a small amount. The second mirror 212b is set so part of the beam is picked-off by the sharp edge of the second mirror 212b. By carefully choosing the spacing between mirrors 212a and 212b and tilting angle, the beam can be sliced into arbitrary numbers of sub-beams, which are arranged horizontally as the exemplified illumination spot 521.

In some embodiments, the first lens group comprises a projection lenses, a collimator, an anamorphic collimator, a circular symmetric lens, or combinations thereof. In some embodiments, the optical slicer is selected from a group consisting of reflective mirrors, a prism, a wedge, and combinations thereof.

After beam was sliced by the optical slicer 212, the illumination area of those sub-beams can selectively be reduced and homogenized through a second lens group (e.g., a beam reducing optics 214) and entered to an interference objective module 3. The spatial and directional distribution of the illuminating light beam is modified by the second lens group. The aim is to homogenize the illumination field within the region of interest of the invention device/system, and matching the shapes of the illumination field and the region of interest of the device/system. For example, to generate a more uniform narrow strip-shaped illumination field. In some embodiments, a polarization beam splitter 22 and quarter wave plate 23 are placed between the second lens group (e.g., the beam reducing optics 214) and the interference objective module 3. In certain embodiments, the beam reducing optics 214 comprises a first beam reducing lens 214a configured to focus the sub-beams sliced by the optical slicer 212; and a second beam reducing lens 214b configured to overlap the sub-beams, which are focused by the first beam reducing lens 214a, to each other and focus the resulted sub-beams onto a common plane (e.g., setting to the aperture plane, or back focal plane) of the objective 31. In some examples, the first beam reducing lens 214a and the second beam reducing lens 214b are standard lens or field lens. The sub-beams will be reformed into illumination spots 531, which is condensed in a second direction perpendicular to the first direction.

In some embodiments, the optical slicer comprises two parallel reflective mirrors, each of them with one sharp edge. In some embodiments, the beam reducing optic is configured to focus the sub-beams sliced by the optical slicer by a first beam reducing lens and overlap the sub-beams to each other by a second beam reducing lens so as to focus the resulted sub-beams onto a common plane of an objective in the interference objective module.

The interference objective module 3 is configured to overlap sub-beams into a uniform output beam to illuminate on a sample. The interference objective module 3 contains an interference component 32. When backscattered light is collected from the sample, interference signals are generated through the interference component 32. After the interference signals transmitted through a quarter wave plate 23 and polarized beam splitter 33, projected by a projection lens 24, and further reflected by a reflective mirror 25, those interference signals will be detected by the detector 24 and turn into an interference image to show the sample's structure. In some examples, the detector can be a two-dimensional (2D) camera/detector, so that the present interference system can be applied in line (B-scan) or wild filed (E-scan) interference scanning.

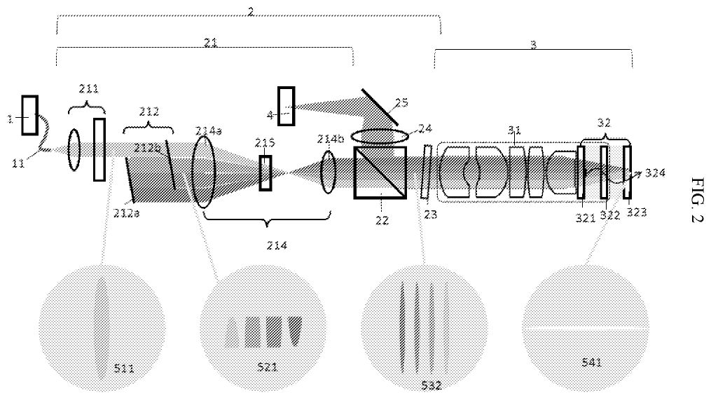

To further enhance the illumination intensity projected on the sample, in some embodiments, the etendue squeezing component 21 further comprises a beam expander 215 to stretch/expand the sub-beams in the first direction as shown in FIG. 2 and the illumination spot 532. In some examples, the beam expander 215 is a concave lens, or the like. The illumination intensity will depend on the squeezing ratio of N. There are two conditions the beam expander 215 is preferably fitted in: (1) the chief rays of sub-beams 521 are (loosely) overlap to each other after the beam reducing optics 214; (2) each sub-beam is focused on a common plane, and the plane is set to the aperture plane or back focal plane of the objective 31 of the interference objective module 3.

In some embodiments, the etendue squeezing component further comprises a beam expander configured to expand the sub-beams processed by the first beam reducing lens. In certain embodiments, the beam expander is a concave lens.

In some embodiments, the optical module 2 further comprises a switch (not shown) to change the output illumination field projected on the sample from a line of illumination field (for B-scan) to an area of illumination filed (for E-scan). The switch can be set between the beam expander 215 and the second beam reducing optics 214b. The switch can also be set between the second beam reducing optics 214b and the polarization beam splitter 22. In certain embodiments, the beam expander 215 is functioned as a switch to change the illumination filed between line illumination field and area illumination field by moving its position toward the position of the first beam reducing optics 214a. A person skilled in the art can readily choose suitable switch in accordance with the practice of the present invention to achieve switching the illumination field between line and area, so as to switch modes of B-scan and E-scan for the illumination measurement.

In some embodiments, the etendue squeezing component further comprises a switch to change the output illumination field projected on the sample from a line of illumination field to an area of illumination filed. In certain embodiments, said switch is placed between the beam expander and the second beam reducing optics, or between the second beam reducing optics and the polarization beam splitter. In certain embodiments, said switch is the beam expander configured to move its position toward the position of the first beam reducing optics from the position of the beam expander.

In some embodiments, the interference objective module 3 comprises an objective 31 and an interference component 32 configured to generate interference signals during the measurement. As illustrated in the figures disclosed herein, in some embodiments, the interference component 32 comprises a first glass plate 321 coated with a reflective mirror 324; a second glass plate 322, and a third glass plate 323, wherein the reflective mirror 324 is coated to generate a reference arm and produce interference with the backscattered light of the sample. In an example, the reflective mirror 324 has a shape of linear parallel to the line of the light 541, or has a shape of circle. The reflective mirror 324 can also comprises a black spot on the opposite side of the first glass plate at a position corresponding to the reflective mirror 324. In some examples, the second glass plate 322 has a refractive ratio of about 5% to 30%, preferably 10% to 20%, or any other suitable ratio as needed based on the condition. The third glass plate 323 is fully transparent for fitting with sample allowing the illumination light projected on the sample.

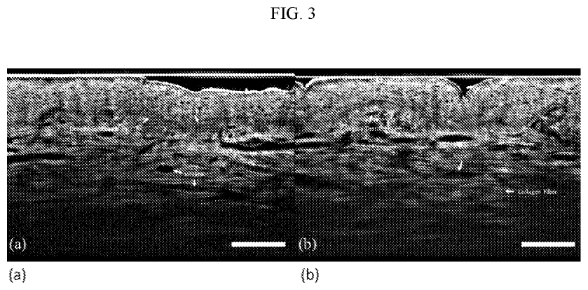

In some embodiments, the light source 1 is an amplified spontaneous emission light source, a super luminescent diode (SLD), a light emitting diode (LED), a broadband super continuum light source, a mode-locked laser, a tunable laser, a Fourier-domain Mode-locking light source, an optical parametric oscillator (OPO), a halogen lamp, a Ce3+:YAG crystal fiber light source, a Ti3+:Al2O3 crystal fiber light source, a Cr4+:YAG crystal fiber light source, or combinations thereof. In certain embodiments, light source 1 is a Ce3+:YAG crystal fiber light source, a Ti3+:Al2O3 crystal fiber light source, a Cr4+:YAG crystal fiber light source, or combinations thereof. In certain embodiments, the light source 1 is a Ti3+:Al2O3 crystal fiber light source. In some examples, the light source 1 can be a small etendue light source of V number around 60. With the present interference system, interference image scanning rate is increased, and the image quality is improved at the same time as illustrated in FIG. 3. FIG. 3(a) and (b) are B-scan interference images of normal human skin with squeezing ration of 6 (N=6). As such, for example, the fine structure of collagen fibers in both papillary and reticular dermis, the cross-sectional orientation of keratinocytes, some arrangement of basal cells, and distribution of melanin near the junction can be easily identified.

In a general illumination system, most of the light will be blocked by a reference mirror if conventional Kohler illumination is applied. In accordance with the practice of the present invention, the invention interference system can selectively offset the beams illuminated on the sample to avoid the light blocked by the reference mirror, so as to avoid linear artifact on the interference image.

In some embodiments, the interference objective module is a Mirau type interference objective module, a Michelson type interference module, or a Mach-Zehnder interference objective module. In certain embodiments, the interference objective module is a Mirau type interference objective module.

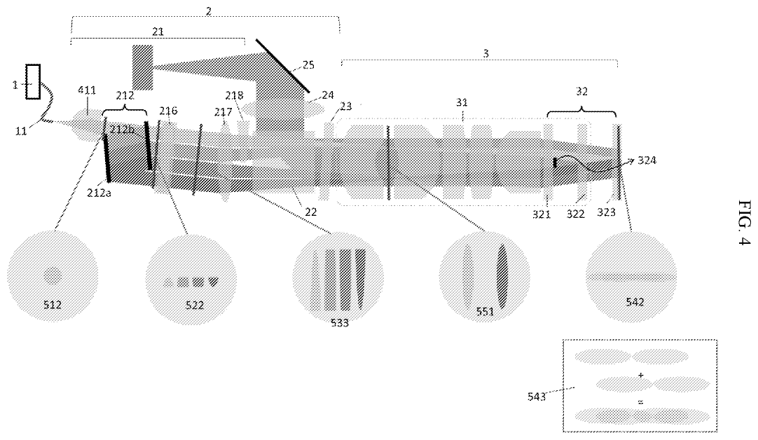

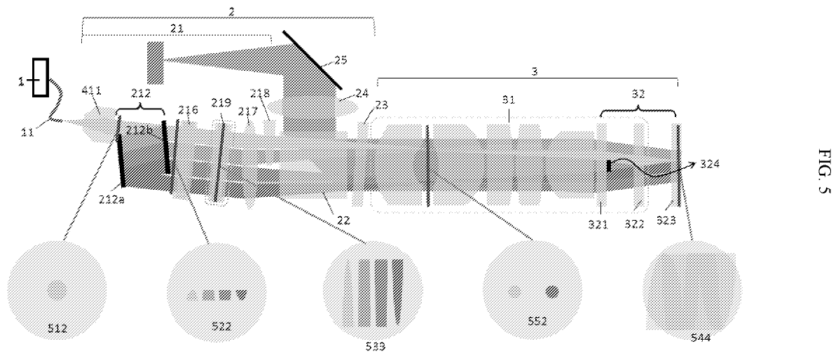

In some embodiments exemplified by FIG. 4 and FIG. 5, the first lens group 411 is an anamorphic collimator (e.g., a convex lens or a cylindrical lens) making the spot size along a first direction is larger comparing to a second direction perpendicular to the first direction. In some embodiments, the first lens group 411 is composed of circular symmetric lens(es), making the illumination filed 512 a circle. When the circle beam process into the optical slicer 212, sub-beams will be generated into a shape shown as 522.

In yet another embodiment of the present invention illustrated in FIG. 4, a second lens group comprising a beam steering element is used in the system/device with an illumination filed in circle. In some embodiment, the etendue squeezing component 21 comprises a second lens group comprising a beam expander, a field lens and a beam steering element to homogenize the sub-beams. The spatial and directional distribution of the illuminating light beam is modified/manipulated by the second lens group. The aim is to homogenize the illumination field within the region of interest, and matching the shapes of the illumination field with the region of interest of the device/system. As a result, to generate a more uniform narrow strip-shaped illumination field. In certain embodiment, the etendue squeezing component 21 is consisting of a second lens group comprising a beam expander, a field lens and a beam steering element to homogenize the illumination fields. The beam expander 216 is configured to stretch/expand the sub-beams 522 in the first direction to provide sub-beams 533. In some embodiments, the beam expander 216 is a negative cylindrical lens. A person skilled in the art can chose an appropriate optical lens to achieve the same function. Instead of directly projecting the field onto a sample, a beam steering element 218, in some embodiments, is placed between a field lens 217 and the polarized beam splitter 22 to generate two illumination fields. The beam steering element 218 is configured to adjust illumination angle of a part of sub-beams 521 to separate the sub-beams 522 into at least two illumination fields. In some embodiments, the beam steering element 218 is selected from a group consisting of a wedge, a prism, and combinations thereof; or the like. The purpose of this arrangement is to avoid the central obscuration of the reflective mirror 324, and illuminate the sample in a symmetrical style at the same time. Each of the illumination field is formed by multiple sub-beams (e.g., two sub-beams are exemplified in FIG. 4), and the illumination field is not uniform. In order to improve the uniformity of the illumination field, the two illumination fields 551 are overlapped near 323 with a small lateral offset to generate a more uniform illumination field 542. The spatial relationship of the two illumination fields 551 and the resulted uniform illumination field 542 are illustrated in 543. In such arrangement, the beam steering angle of the beam steering element 218 could be less than half of the whole bundle beam's converging angle. As such, the beam passing through the beam steering element 218 is not parallel to the beam without passing through the beam steering element 218.

In some embodiments, the etendue squeezing component comprises a first lens group configured to reduce light divergence angle from the light source and project to an optical slicer wherein said optical slicer slices the processed beam to at least two sub-beams to be further processed by a second lens group before entering to said interference objective module via a polarization beam splitter. In certain embodiments, said second lens group comprises a beam expander, a field lens and a beam steering element to homogenize the sub-beams. In certain embodiments, the beam expander expands the sub-beams and projects to a field lens and then processed by a beam steering element. In certain embodiments, the first lens group is an anamorphic collimator making the illumination filed a circle. In certain embodiments, the beam expander is a negative cylindrical lens. In some embodiments, the beam steering element is configured to adjust illumination angle of a part of sub-beams to separate the sub-beams into at least two illumination fields. In certain embodiments, the beam steering element is selected from a group consisting of a wedge, a prism, and combinations thereof. In certain embodiments, the beam steering element is placed between the field lens and a polarized splitter.

In yet another embodiment as shown in FIG. 5 provides an example showing how to process E-scan interference measurements via an invention system/device. The etendue squeezing component 21 comprises a beam expander 216 configured to stretch/expand the sub-beams 522 in the first direction to provide sub-beams 533. In some embodiments, the beam expander 216 is a negative cylindrical lens. A person skilled in the art can chose an appropriate optical lens to achieve the same function.

Instead of directly projecting the field onto a sample, a beam steering element 218, in some embodiments, is placed after the field lens 217, to generate two illumination fields. A positive cylindrical lens 219 is placed before a beam steering element 218 to input the illumination fields to the interference objective module 3 changing a line of illumination field to an area of illumination field as shown in FIG. 5. The positive cylindrical lens 219 can be positioned between the beam expander 216 and the field lens 217. When the positive cylindrical lens 219 is placed, the sub-beams passing through the beam steering element 218 (e.g., a wedge plate) will become two circle spots 552, and an illumination area (as output spot 544) will be illustrated on the sample to process E-scan interference measurement.

In some embodiments, the etendue squeezing component further comprises a positive cylindrical lens placed between the beam expander and the field lens. In certain embodiments, the positive cylindrical lens inputs the sub-beams passing through the beam steering element to two circle spots changing a line of illumination field to an area of illumination field.

By focusing the light emitted from a multimode fiber into a narrow line, the illumination intensity increases, and the spatial coherence of illumination reduces. In some embodiments, when a detector is a two-dimensional camera (e.g., PhotonFocus MV1-D1024E-160-CL) of 200,000-electrons full well capacity and low quantum efficiency at near infrared (<20%), the camera hits saturation at 0.02 ms at 10-mW optical power level, and >20 kHz camera frame rate can be achieved with 1024×3 pixel format, which is close to the upper limit of camera pixel clock.

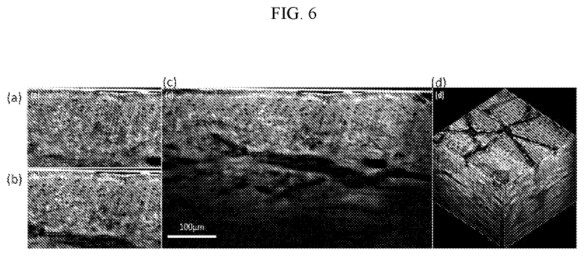

In accordance with the practice of the present invention, an exemplary device/system with etendue squeezing illumination provides the special coherence area to be about 1 μm², which is roughly equal to the spatial resolution of the present interference system. As such, the most of coherent crosstalk is rejected, and B-scan image quality is apparently improved as shown in FIG. 6. FIG. 6(a) shows a B-scan image with high-spatial-coherence illumination. FIG. 6(b) shows a B-scan image with low-spatial-coherence illumination. It is clearly shown that the visibility of nucleui, clusters of melanin, dermal-epidermal junction and papilary strcuture of upper dermis singnificantly improved.

FIG. 6(c) shows a B-scan image with ˜5-μm virtual slice thickness acquired with low-spatial-coherence illumination, which is closer to a histological slice and speckle contrast to further reduced. FIG. 6(d) shows a three-dimensional image acquired with wide-field illumination where 219 is used so a volumetric imaging can be performed.

In some embodiments as illustrated in FIG. 5, when the positive cylindrical lens 219 is positioned, it is functioned as a switch for changing modes between B-scan and E-scan. When the switch 219 turns on (i.e., cause to place between the beam expander 216 and filed lens 217), the sub-beams passing through the beam steering element 218 becomes two circle spots shown in 552, and an illumination area (as an output spot 544) will be illustrated on the sample to process E-scan interference measurement.

In some embodiments, by using the present interference system, B-scan with ˜5μm virtual slice thickness will be approached to a histological slice. Furthermore, speckle contrast will be further reduced. By inserting switch 219 (e.g., a positive cylindrical lens), wide-field illumination can be generated, and volumetric imaging can be performed, which is shown in FIG. 6(d).

The present invention also provides a method of detecting interference signals to improve the interference image quality, which comprises: providing a beam from a light source; reducing light divergence angle of the beam from the light source by a first lens group; slicing the beam to at least two sub-beams by an optical slicer; homogenizing the sub-beams and matching the shapes of the illumination field with the region of interest and projecting on a sample; and detecting interference signals backscattered from the sample.

In some embodiments provide a method of detecting interference signals comprising: providing a beam from a light source; reducing light divergence angle of the beam from the light source by a first lens group; slicing the beam to at least two sub-beams by an optical slicer; homogenizing the sub-beams and matching the shapes of the illumination field with the region of interest by focusing the sub-beams sliced by the optical slicer and overlapping illumination fields of the resulted sub-beams and projecting on a sample; and detecting interference signals backscattered from the sample.

In certain embodiments, the illumination filed is a line of illumination field or an area of illumination filed. In certain embodiments, the method further comprises switching the line of illumination field to the area of illumination field by a switch. In certain embodiments, said switch is a positive cylindrical lens.

Because the method and the apparatus of the present invention applying etendue squeezing method/means to slice light into sub-beams and overlap them into an illumination filed, fewer light photons will be wasted. As for this reason, the intensity of illumination beam on the sample will be increased, the scanning rate will be increased, and the image quality will be improved. In addition, by evenly overlapping illumination filed of sub-beams, the uniform illumination will improve interference image quality to show more detail structures of skin image with few artifacts or speckles.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What claimed is:

1. An interference device comprising:
a light source which provides a beam to an optical module via a multimode fiber;
an interference objective module outputs the beam processed by the optical module and collects interference signals from a sample; and a detector which detects the interference signals from the interference objective module wherein the optical module comprises an etendue squeezing component configured to slice the beams to at least two sub-beams and homogenize the sub-beams to an illumination field and match the shapes of the illumination field with a region of interest.

2. The interference device of claim 1, wherein the etendue squeezing component comprises a first lens group configured to reduce light divergence angle of the beam from the light source and project to an optical slicer wherein said optical slicer slices the processed beam to at least two sub-beams to be further processed by a second lens group before entering to said interference objective module via a polarization beam splitter.

3. The interference device of claim 2, wherein the second lens group comprises a beam reducing optics.

4. The interference device of claim 3, wherein the number of sub-beams is decided by a squeezing ratio of N.

5. The interference device of claim 4, wherein the squeezing ratio of N is 2 to 16, 2 to 14, 2to 10, or 2 to 8.

6. The interference device of claim 2, wherein the first lens group comprises a projection lenses, a collimator, an anamorphic collimator, a circular symmetric lens, or combinations thereof.

7. The interference device of claim 2, wherein the optical slicer is selected from a group consisting of reflective mirrors, a prism, a wedge, and combinations thereof.

8. The interference device of claim 7, wherein the optical slicer comprises two parallel reflective mirrors, each of them with one sharp edge.

9. The interference device of claim 3, wherein the beam reducing optic is configured to focus the sub-beams sliced by the optical slicer by a first beam reducing lens and parallel the sub-beams to each other by a second beam reducing lens so as to focus the resulted sub-beams onto a common plane of an objective in the interference objective module.

10. The interference device of claim 9, wherein the etendue squeezing component further comprises a beam expander configured to expand the sub-beams processed by the first beam reducing lens.

11. The interference device of claim 10, wherein a beam expander is a concave lens.

12. The interference device of claim 1, wherein the etendue squeezing component comprises a first lens group configured to reduce light divergence angle of the beam from the light source and project to an optical slicer wherein said optical slicer slices the processed beam to at least 2 sub-beams to be further processed by a second lens group before entering to said interference objective module via a polarization beam splitter.

13. The interference device of claim 12, wherein said second lens group comprises a beam expander, a field lens and a beam steering element to homogenize the sub-beams.

14. The interference device of claim 13, wherein the beam expander expands the sub-beams and projects to a field lens and then processed by a beam steering element.

15. The interference device of claim 12, wherein the first lens group is an anamorphic collimator making the illumination filed a circle.

16. The interference device of claim 14, wherein the beam expander is a negative cylindrical lens.

17. The interference device of claim 14, wherein the beam steering element is configured to adjust illumination angle of a part of sub-beams to separate the sub-beams into at least two illumination fields.

18. The interference device of claim 14, wherein the beam steering element is selected from a group consisting of a wedge, a prism, and combinations thereof.

19. The interference device of claim 14, wherein the beam steering element is placed between the field lens and a polarization beam splitter.

20. The interference device of claim 14, wherein the etendue squeezing component further comprises a positive cylindrical lens placed between the beam expander and the field lens.

21. The interference device of claim 20, wherein the positive cylindrical lens inputs the sub-beams passing through the beam steering element to two circle spots changing a line of illumination field to an area of illumination field.

22. The interference device of claim 9, wherein the etendue squeezing component further comprises a switch to change the output illumination field projected on the sample from a line of illumination field to an area of illumination filed.

23. The interference device of claim 22, wherein said switch is placed between the beam expander and the second beam reducing optics, or between the second beam reducing optics and the polarization beam splitter.

24. The interference device of claim 23, wherein said switch is the beam expander configured to move its position toward the position of the first beam reducing optics from the position of the beam expander.

25. The interference device of claim 3, wherein the interference objective module is configured to overlap illumination fields of the sub-beams into an output illumination field.

26. The interference device of claim 1, wherein the detector is a 2D detector.

27. The interference device of claim 1, the light source is an amplified spontaneous emission light source, a super luminescent diode (SLD), a light emitting diode (LED), a broadband super continuum light source, a mode-locked laser, a tunable laser, a Fourier-domain Mode-locking light source, an optical parametric oscillator (OPO), a halogen lamp, a Ce3+:YAG crystal fiber light source, a Ti3+:Al2O3 crystal fiber light source, and a Cr4+:YAG crystal fiber light source, or combinations thereof.

28. The interference system of claim 27, wherein the light source is a Ce3+:YAG crystal fiber light source, a Ti3+:Al2O3 crystal fiber light source, and a Cr4+:YAG crystal fiber light source or combinations thereof.

29. The interference device of claim 1, wherein the interference objective module comprises an interference component configured to generate interference signals during the measurement.

30. The interference system of claim 1, wherein interference objective module is a Mirau type interference objective module, a Michelson type interference module, or a Mach-Zehnder interference objective module.

31. A method of detecting interference signals comprising:
providing a beam from a light source;
reducing light divergence angle of the beam from the light source by a first lens group;
slicing the beam to at least two sub-beams by an optical slicer;
homogenizing the sub-beams and matching the shapes of the illumination field with the region of interest and projecting on a sample; and
detecting interference signals backscattered from the sample.

32. The method of claim 31, wherein the illumination filed is a line of illumination field or an area of illumination filed.

33. The method of claim 32, wherein the method further comprises switching the line of illumination field to the area of illumination field by a switch.

34. The method of claim 33, wherein said switch is a positive cylindrical lens.
